(19) United States
(12) Patent Application Publication
(10) Pub. No.: US 2006/0154260 A1
(43) Pub. Date: Jul. 13, 2006

(54) Sample preparation methods for diagnostic analyses

(76) Inventors: William Mark Barbour, Kingsport, TN (US); Spencer Erich Hochstetler, Kingsport, TN (US)

Correspondence Address: Jonathan D. Wood;Eastman Chemical Company, P.O. Box 511, Kingsport, TN, 37662-5075, US

(21) Appl. No.: 11/031,657

(22) Filed: Jan. 7, 2005

(51) Int. Cl. C12Q 1/68; G01N 33/554; G01N 33/569

(52) U.S. Cl. 435/6; 435/7.32

(57) ABSTRACT

A method for determining the presence or amount of an analyte in a food sample is provided. The method includes the steps of contacting a food sample containing polymer or lipid with an enzyme capable of degrading the polymer or lipid, and analyzing at least a portion of the enzyme-treated sample using a material comprising micro-channels and target-specific capture elements to determine the presence or amount of the analyte.

### FIELD OF THE INVENTION

The invention generally relates to methods for determining the presence or amount of an analyte in a food sample containing a polymer and/or a lipid.

### BACKGROUND OF THE INVENTION

Significant effort and resources have been devoted to developing rapid diagnostic methods for the food, medical, environmental, and veterinary industries. The food industry, for example, needs rapid microbial testing to determine whether a lot of product has been contaminated.

Rapid microbiological methods, such as nucleic acid probe hybridization and immunoassay, have been developed to meet this need. However, some of these methods are very sensitive as to the kind of sample it will reliably analyze, and relatively little effort has been devoted to preparing samples that are not readily conducive for analysis by these methods. For example, there has been little, if any, discussion in the art on how to treat highly heterogeneous samples, such as ground beef that has been homogenized in an enrichment medium or a diluent solution, so that they can be reliably analyzed by these methods. Such samples often have multiple phases and multiple components, such as proteins, lipids, and carbohydrates. The presence of multiple phases and components in the sample often reduces the sensitivity of the assay even if the sample is filtered or separated before analysis.

It would, therefore, be desirable to have a method for reliably determining the presence or amount of an analyte in a heterogeneous food sample. In particular, it would be desirable to have a method for pretreating a highly heterogeneous food sample to make it more conducive for reliable analysis using diagnostic methods.

### SUMMARY OF THE INVENTION

The invention relates to a method for determining the presence or amount of an analyte in a food sample comprising polymer or lipid. In one embodiment, the process comprises:

(a) contacting a food sample comprising polymer or lipid with an enzyme capable of degrading the polymer or lipid to form an enzyme-treated sample, wherein the polymer, if present, is from the source of the sample; and

(b) analyzing at least a portion of the enzyme-treated sample using a material comprising micro-channels and target-specific capture elements to determine the presence or amount of an analyte in the portion of sample.

In another embodiment, the food sample is in liquefied form. The liquefied sample may contain other additives, including a solvent, a pH buffer, a salt, a surfactant, a nutrient for the analyte, or combinations thereof.

In another embodiment, the process comprises:

(a) contacting a liquefied food sample comprising polymer or lipid with an enzyme capable of degrading said polymer or lipid to form an enzyme-treated sample, wherein the polymer, if present, is from the source of the sample;

(b) heating the enzyme-treated sample;

(c) cooling the heated sample; and

(d) analyzing at least a portion of the cooled sample using a material comprising micro-channels and target-specific capture elements to determine the presence or amount of an analyte in the portion of sample.

### DETAILED DESCRIPTION OF THE INVENTION

It has been surprisingly discovered that food samples that have previously been difficult to perform rapid diagnostic analyses on can be treated with a suitable enzyme to render them more susceptible to those analyses.

As used herein, “food sample” means a sample of a food or food product, or a sample from a surface that may have been in contact with a food or food product. Examples of food samples include processed or unprocessed meats (such as beef, poultry, pork, fish, shellfish, etc.), vegetables, fruits, nuts, eggs, diary, spices, breads, chocolates, etc. Food samples also include swabs, sponges or wipes of a surface that may have been in contact with a food or food product. Surfaces that may have come in contact with food or food product include counter tops, packaging, transporting equipment, processing equipment, and in proximity to a food processing area, etc.

The food samples will typically contain a polymer, more specifically, a biopolymer, or a lipid, or both. Examples of biopolymers include protein, polysaccharide, and nucleic acid.

The method of the invention includes the step of contacting the food sample with an enzyme capable of degrading the polymer or lipid in the food sample to form an enzyme-treated sample. If the food sample that is contacted with the enzyme contains polymer, then the polymer should be from the source of the food product as opposed to an exogenous polymer.

The enzyme used in the contacting step may be any enzyme that is capable of degrading the polymer and/or lipid in the food sample. The particular enzyme or enzymes used will depend on the polymer and/or lipid present in the food sample. The enzyme should match the polymer and/or lipid present in the sample. Examples of suitable enzymes include protease, lipase, nuclease, glycosidase, or combinations thereof.

The enzyme is added in an amount sufficient to degrade the polymer and/or lipid in the sample. While the amount will vary depending on the amount of sample being employed, the enzyme is typically used in sufficient concentration to digest the polymer or lipid within approximately 2-60 minutes.

The enzyme may be contacted with the food sample by any method known in the art. For example, the enzyme may be contacted with the food sample by mixing a liquid containing the enzyme with the food sample. Alternatively, the enzyme may be in dry form and mixed directly into the food sample.

In a preferred embodiment, the food sample is liquefied. By “liquefied,” it is meant that the sample has a sufficient amount of liquid phase, at room temperature and pressure, so that it can be treated like a liquid for handling and testing purposes. For samples that are not liquefied in their native state, they may be made liquefied by methods known in the art such as, for example, by pureeing or by mixing the sample with a suitable liquid. A suitable liquid is one that would minimally interfere, if at all, with the particular analyte detection method to be used for analysis. Examples of suitable liquids include solvents such as water. The solvents may contain one or more additives known in the art such as pH buffers, salts, surfactants, and nutrients.

Examples of additives include pH buffers such as phosphate, Tris, MOPS, and HEPES; salts such as sodium chloride, potassium chloride, and magnesium chloride; classes of surfactants such as Tween® and Triton® (e.g. Tween® 20, Tween® 80, Triton®-X100, etc.); and nutrients including sugars such as glucose, sucrose, and lactose, and protein extracts such as peptone and tryptone.

The polymer or lipid in the food sample may undergo cleavage during the contact step.

Before the enzyme contacting step, the food sample may optionally be homogenized by blending or stomaching, typically for 0.5 to 2 minutes, and incubated at temperatures typically ranging from 20 to 45° C. for 2 to 40 hours.

After mixing the enzyme with the food sample, the sample may optionally be heated (1) to render innocuous any pathogens that may be present in the sample; (2) to allow the enzyme to pass through its optimal temperature for activity; (3) to make the target analyte more antigenic for increased binding with antibodies in the assay; (4) to inactivate or degrade the activity of the enzyme, or any combinations thereof. The sample may be heated at a temperature ranging from room temperature up to approximately 121° C. Preferably, the sample is heated at 105° C. The sample may be heated for 0 to 30 minutes. Preferably, the sample is heated for 5 to 10 minutes.

Following the heating step, the sample may optionally be cooled at room temperature or in a cooling apparatus for 0 to 10 minutes. Examples of cooling apparatuses include a water bath, an ice bath, and a cooling block.

After the enzyme contacting step, at least a portion of the enzyme-treated sample may be analyzed to determined the presence and/or amount of an analyte in the sample portion. The method of the invention can be used to detect any analyte that has been analyzed using assaying techniques employing materials comprising micro-channels and target-specific capture elements, or that is analyzable by such techniques.

As used herein, “micro-channels” refer to channels having a sub-millimeter diameter. The channels may be randomly oriented like those in a synthetic or naturally occuring porous membrane (e.g. typical immunochromatographic mambranes). Alternately, the channels may be “monolithic” such as those found on microfluidic devices. “Target-specific capture elements” refer to chemicals or biochemicals having a selective affinity to the target. “Target” refers to analytes or parts thereof, or complexes of analytes or parts thereof with a label. Examples of labels include antibodies, antibody conjugates, and nucleic acids.

Various assaying techniques suitable for use in the present invention include immunoassays and nucleic acid tests. Immunoassays include microfluidic immunoassays such as those described in Analytical Chemistry, Vol. 74, No. 12, pp. 2637-2652 (Jun. 15, 2002) and Analytical and Bioanalytical Chemistry, Vol. 337, No. 3, pp. 556-569 (2003), and immunochromatographic techniques such as those described in U.S. Pat. Nos. 6,485,982 and 6,534,320 including but not limited to visual, fluorescence, chemiluminescence, or reflectance methods of detection. Preferred are immunochromatographic assays in which the result is not readily detectable visually. Among these non-visual immunochromatographic assays, preferred are assays based on the magnetic properties of localized magnetically labeled analytes. Such assays are described, for example, in U.S. Pat. No. 6,046,585 and its progeny.

Examples of nucleic acid tests include polymerase chain reaction (PCR), ligase chain reaction (LCR), and DNA or RNA hybridization including techniques employing microfluidics such as the Caliper LifeSciences LabChip system or the Agilent Technologies Lab-on-a-Chip.

The analytes that can be detected by the present invention include either viruses, cells, or portions thereof including their excretions and metabolites. The cells can be either prokaryotic or eukaryotic cells. Eukaryotic cells include, for example, growth factor dependent cells, fetal cells, bone marrow derived cells, blood derived cells and tumor derived cells. Additional examples includes eukaryotic microorganisms, such as yeast, fungi, protozoan and nematode cells. Prokaryote cells include, for example, pathogenic and non-pathogenic bacteria. Microorganisms that can be detected using the method of the present invention include, but are not limited to, bacteria, viruses, fungi, protozoans, and nematodes. For example, the bacteria can be Escherichia coli, Salmonella, Campylobacter, Legionella, Clostridium, Pseudomonas, Listeria, Staphylococcus, Bacillus, Shigella, Mycobacteria, Bordetella, Streptococcus, etc. The viruses include, for example, but are not limited to, viruses of the families: Poxyiridae, Iridoviridae, Herpesviridae, Adenoviridae, Papovarviridae, and Retroviridae, such as the Acquired Immune Deficiency Syndrome (AIDS) virus, etc. Fungi which can be detected by the method of the present invention include, for example, Aspergillus, Blastomyces, Candida (such as yeast), Coccidioides, Cryptococcus and Histoplasma, etc. The protozoan groups that can be detected by the method of the present invention include, for example, Rhizopoda (e.g., amoeba such as Entamoeba histolytica, and Dientamoeba fragilis), Mastigophora (flagellates) (e.g., Giardia larablia), Ciliatea (ciliates, e.g., Balantidium coli) and Sporozoa (e.g., Isospora, Cryptosporidium).

Preferred analytes are pathogenic organisms such as members of Listeria spp. and Salmonella spp., and serotypes of Escherichia coli.

The invention is further illustrated by the following examples.

### EXAMPLES

### Example 1

Increased signal above background after sample pre-treatment.

An enrichment culture was prepared by blending a raw ground beef sample in a proprietary enrichment broth at a ratio of 1:10. The enrichment was incubated at 42° C. for approximately 7 hours. An aliquot of the enrichment was inoculated with a diluted broth culture of E. coli O157:H7 to yield a concentration of approximately 1000 cells per mL of enrichment. A second aliquot was prepared as an uninoculated control (blank). The inoculated and blank samples were further dispensed at 1.2 mL volumes into 2 mL screw-cap tubes. A protease solution at 200 or 20 mg/mL concentration was added to the tubes at volumes of 0, 10, and 100 μL to generate samples with 0, 0.2, 2, and 20 mg of protease per tube. The samples were incubated at room temperature for 10 minutes, then heated in a 105° C. heating block for 10 minutes, then cooled at room temperature for 5 minutes. Next, 150 μL of each samples was mixed with 20 μL of an anti-E. coli O157 antibody-magnetic particle conjugate suspension. One hundred and fifty microliters of the resulting mixture was transferred to an immunochromatographic device. After development for 25 minutes, the devices were placed in a reader capable of determining the accumulation of target-conjugate complex at an anti-E. coli O157 “test” line on the assay device and the accumulation of conjugate at an anti-(anti-E. Coli O157) “control” line.

Table 1 below shows the data for signal at the test and control lines on each device.

TABLE 1

| | Amount of protease added per tube | | | | | | | |
|---|---|---|---|---|---|---|---|---|
| Inocu- | No protease | | 20 mg | | 2 mg | | 0.2 mg | |
| lation | | Con- | | Con- | | Con- | | Con- |
| Cells/mL | Test | trol | Test | trol | Test | trol | Test | trol |
| 1000 | 0.7 | 435.4 | 38.6 | 1033.0 | 29.8 | 885.1 | 22.1 | 1244.6 |
| 1000 | 3.8 | 539.1 | 36.4 | 1045.1 | 31.2 | 823.0 | 32.0 | 987.0 |
| Blank | 5.6 | 348.5 | 18.3 | 1143.2 | 6.5 | 852.7 | 8.2 | 928.2 |
| Blank | 0.0 | 428.1 | 18.7 | 1118.4 | 5.0 | 811.3 | 6.3 | 1077.6 |

As seen in Table 1, adding protease to a food enrichment increased the signal strength for detection of a bacterial analyte using an immunochromatographic assay. Further, there was a significant increase in the ratio of test signal arising from the inoculated samples to the test signal arising from the uninoculated samples.

### Example 2

Increased signal after treatment with five different proteases.

An enrichment culture was prepared by blending a raw ground beef sample in a proprietary enrichment broth at a ratio of 1:10. The enrichment was incubated at 42° C. for approximately 7 hours. An aliquot of the enrichment was inoculated with a diluted broth culture of E. coli O157:H7 to yield a concentration of approximately 1000 cells per mL of enrichment. A second aliquot was prepared as an uninoculated control (blank). The inoculated and blank samples were further dispensed at 1.2 mL volumes into 2 mL screw-cap tubes. A 20 μL volume of one of five protease solutions, each at 200 mg/mL concentration (or for Promod 439L, the concentration supplied by the manufacturer), was added to a portion of the tubes to generate samples with either 0 or 2 mg of protease per tube. The samples were mixed briefly, then heated in a 105° C. heating block for 10 minutes, then cooled at room temperature for 5 minutes. Next, 150 μL of each samples was mixed with 20 μL of an anti-E. coli O157 antibody-magnetic particle conjugate suspension. One hundred and fifty microliters of the resulting mixture was transferred to an immunochromatographic device. After development for 25 minutes, the devices were placed in a reader capable of determining the accumulation of target-conjugate complex at an anti-E. coli O157 “test” line on the assay device and the accumulation of conjugate at an anti-(anti-E. Coli O157) “control” line.

Table 2 shows the data for signal at the test (T) and control (C) lines on each device.

TABLE 2

| | Protease (P) used | | | | | | | | | | | |
|---|---|---|---|---|---|---|---|---|---|---|---|---|
| | | | P1 | | P2 | | P3 | | P4 | | P5 | |
| | | | (Amano | | (Amano | | (Biocatalyst | | (Biocatalyst | | (Biocatalyst | |
| Inoculation | None | | Protease P) | | Protease N) | | s Promod 24P) | | s Promod 194P) | | s Promod 439L) | |
| Cells/mL | T | C | T | C | T | C | T | C | T | C | T | C |
| 1000 | 5.4 | 582.1 | 34.7 | 1137.5 | 29.1 | 1093.1 | 33.3 | 1133.5 | 37.0 | 1157.8 | 24.4 | 1154.9 |
| 1000 | 3.3 | 493.6 | 33.6 | 956.9 | 29.3 | 1007.5 | 28.6 | 1396.5 | 38.2 | 1139.4 | 24.8 | 1179.9 |
| Blank | 0.0 | 415.3 | 15.5 | 1214.1 | 8.9 | 1118.1 | 12.9 | 1402.7 | 12.0 | 1248.5 | 6.9 | 1074.3 |
| Blank | 0.0 | 475.2 | 19.0 | 1041.7 | 11.1 | 1094.8 | 9.6 | 1254.2 | 11.4 | 1534.6 | 10.5 | 1457.8 |

As seen in Table 2, adding different proteases to a food enrichment increased the signal strength for detection of a bacterial analyte using an immunochromatographic assay. Further, there was a significant increase in the ratio of test signal arising from the inoculated samples to the test signal arising from the uninoculated samples.

### Example 3

Time course for visual observation of sample clearing due to protease treatment.

A beef sample enrichment was prepared as in the above examples then incubated approximately 20 hours at 42° C. One milliliter of sample was placed in a series of tubes to which a range of volumes of protease was added. The tubes were then heated for five minutes in a 105° C. heating block. At each minute the samples were briefly removed from the block to allow observation of sample turbidity. The turbidity was scored based on whether the sample was decidedly clear compared to the no protease control sample. The data is shown in Table 3.

TABLE 3

| | Clearing of sample after indicated minutes of | | | | |
|---|---|---|---|---|---|
| Protease | incubation at 105° C. | | | | |
| volume (μL) | 1 | 2 | 3 | 4 | 5 |
| 0 | No | No | No | No | No |
| 16.0 | No | Yes | Yes | Yes | Yes |
| 7.0 | No | Yes | Yes | Yes | Yes |
| 2.5 | No | Yes | Yes | Yes | Yes |
| 1.0 | No | Partial | Partial | Yes | Yes |

In addition to an increase in signal seen in Examples 1 and 2, as seen in Table 3, the activity of the protease can be observed visually as a clearing, or reduction in turbidity of a food sample. This is an improvement since sample turbidity can obscure pipettes when attempting to transfer samples and can also cause physical blocking in sample manipulations such as pipetting and immunochromatography.

We claim:

1. A method for determining the presence or amount of an analyte in a food sample comprising polymer or lipid, said process comprising:
(a) contacting a food sample comprising polymer or lipid with an enzyme capable of degrading said polymer or lipid to form an enzyme-treated sample, wherein said polymer, if present, is from the source of the sample; and
(b) analyzing at least a portion of said enzyme-treated sample using a material comprising micro-channels and target-specific capture elements to determine the presence or amount of an analyte in the portion of sample.

2. The method according to claim 1, wherein polymer or lipid cleavage occurs during said contacting step.

3. The method according to claim 1, wherein said polymer is a biopolymer.

4. The method according to claim 3, wherein said biopolymer is a protein, polysaccharide, nucleic acid, or combinations thereof.

5. The method according to claim 1, wherein said enzyme is a protease, lipase, nuclease, glycosidase, or combinations thereof.

6. The method according to claim 1, wherein said enzyme is a protease.

7. The method according to claim 1, wherein said analyte is prokaryotic, eukaryotic, or viral.

8. The method according to claim 1, wherein said analyte is a pathogen.

9. The method according to claim 8, wherein said pathogen is a member of Listeria spp. or Salmonella spp., or a serotype of Escherichia coli.

10. The method according to claim 1, wherein said analyzing step comprises immunoassay or nucleic acid testing.

11. The method according to claim 10, wherein said analyzing step comprises immunoassay.

12. The method according to claim 11, wherein said immunoassay comprises a microfluidic immunoassay.

13. The method according to claim 11, wherein said immunoassay comprises immunochromatographic assay.

14. The method according to claim 13, wherein said immunochromatographic assay comprises non-visual assay.

15. The method according to claim 14, wherein said non-visual, immunochromatographic assay comprises an assay of magnetic properties of localized magnetically labeled analyte.

16. A method for determining the presence or amount of an analyte in a liquefied food sample comprising polymer or lipid, said process comprising:
(a) contacting a liquefied food sample comprising polymer or lipid with an enzyme capable of degrading said polymer or lipid to form an enzyme-treated sample, wherein said polymer, if present, is from the source of the sample; and
(b) analyzing at least a portion of said enzyme-treated sample using a material comprising micro-channels and target-specific capture elements to determine the presence or amount of an analyte in the portion of sample.

17. The method according to claim 16, wherein said liquefied food sample comprises a solvent, a pH buffer, a salt, a surfactant, a nutrient for the analyte, or combinations thereof.

18. The method according to claim 16, wherein polymer or lipid cleavage occurs during contacting step (a).

19. The method according to claim 16, wherein said polymer is a biopolymer selected from the group consisting of a protein, a polysaccharide, a nucleic acid, and combinations thereof.

20. The method according to claim 16, wherein said enzyme is a protease, lipase, nuclease, glycosidase, or combinations thereof.

21. The method according to claim 16, wherein said enzyme is protease.

22. The method according to claim 16, wherein said analyte is prokaryotic, eukaryotic, or viral.

23. The method according to claim 16, wherein said analyte is a pathogen.

24. The method according to claim 23, wherein said pathogen is a member of Listeria spp. or Salmonella spp., or a serotype of Escherichia coli.

25. The method according to claim 16, wherein said analyzing step comprises immunoassay or nucleic acid testing.

26. The method according to claim 25, wherein said immunoassay comprises a microfluidic immunoassay.

27. The method according to claim 25, wherein said analyzing step comprises immunochromatographic assay.

28. The method according to claim 27, wherein said immunochromatographic assay comprises non-visual assay.

29. The method according to claim 28, wherein said non-visual immunochromatographic assay is an assay of magnetic properties of localized magnetically labeled analyte.

30. A method for determining the presence or amount of an analyte in a liquefied food sample comprising polymer or lipid, said process comprising:
(a) contacting a liquefied food sample comprising polymer or lipid with an enzyme capable of degrading said polymer or lipid to form an enzyme-treated sample, wherein said polymer, if present, is from the source of the sample;
(b) heating said enzyme-treated sample;
(c) cooling said heated sample; and
(d) analyzing at least a portion of said cooled sample using a material comprising micro-channels and target-specific capture elements to determine the presence or amount of an analyte in the portion of sample.

31. The method according to claim 30, wherein said liquefied sample comprises a solvent, a pH buffer, a salt, a surfactant, a nutrient for the analyte, or combinations thereof.

32. The method according to claim 30, wherein polymer or lipid cleavage occurs during contacting step (a).

33. The method according to claim 30, wherein said polymer is a biopolymer selected from the group consisting of protein, polysaccharide, nucleic acid, and combinations thereof.

34. The method according to claim 30, wherein said enzyme is a protease, lipase, nuclease, glycosidase, or combinations thereof.

35. The method according to claim 34, wherein said enzyme is protease.

36. The method according to claim 30, wherein said analyte is prokaryotic, eukaryotic, or viral.

37. The method according to claim 30, wherein said analyte is a pathogen.

38. The method according to claim 37, wherein said pathogen is a member of Listeria spp. or Salmonella spp., or a serotype of Escherichia Coli.

39. The method according to claim 30, wherein said enzyme-treated sample is heated at least to the enzyme's optimal temperature for activity.

40. The method according to claim 39, wherein said optimal temperature ranges from room temperature to 121° C.

41. The method according to claim 30, wherein said analyzing step comprises immunoassay or nucleic acid testing.

42. The method according to claim 30, wherein said analyzing step comprises immunoassay.

43. The method according to claim 42, wherein said immunoassay comprises a microfluidic immunoassay.

44. The method according to claim 42, wherein said analyzing step comprises immunochromatographic assay.

45. The method according to claim 44, wherein said immunochromatographic assay comprises non-visual assay.

46. The method according to claim 45, wherein said non-visual immunochromatographic assay is an assay of magnetic properties of localized magnetically labeled analyte.
